(19) United States
(12) Patent Application Publication
(10) Pub. No.: US 2008/0311219 A1
(43) Pub. Date: Dec. 18, 2008

(54) Tissue Fragment Compositions for the Treatment of Incontinence

(76) Inventors: Anna Gosiewska, Skillman, NJ (US); Agnieszka Seyda, Edison, NJ (US); Charito S. Buensuceso, North Brunswick, NJ (US); Sridevi Dhanaraj, Raritan, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/133,770

(22) Filed: Jun. 5, 2008

Related U.S. Application Data
(60) Provisional application No. 60/944,266, filed on Jun. 15, 2007.

(51) Int. Cl. A61K 35/34; A61P 13/00

(52) U.S. Cl. 424/548

(57) ABSTRACT

Compositions for the treatment of incontinence are disclosed. More particularly, compositions of viable muscle tissue fragments and a carrier are disclosed. The compositions are useful in the treatment urinary and fecal incontinence.

### FIELD OF THE INVENTION

The invention relates to compositions for the treatment of incontinence. More specifically, the invention relates to compositions comprising viable muscle tissue fragments and a carrier for the treatment of incontinence.

### BACKGROUND OF THE INVENTION

Injuries to soft tissue, for example, vascular, skin, or musculoskeletal tissue, are quite common. Many of these disorders occur in the absence of systemic disease and are a consequence of chronic repetitive low-grade trauma and overuse.

One example of a fairly common soft tissue injury is incontinence. Incontinence is the complaint of any involuntary leakage of urine or feces. It can cause embarrassment and lead to social isolation, depression, loss of quality of life, and is a major cause for institutionalization in the elderly population. There are several types of incontinences including urge incontinence or urge urinary incontinence, stress incontinence or stress urinary incontinence, overflow incontinence, and mixed incontinence or mixed urinary incontinence. Mixed incontinence or mixed urinary incontinence refers to the case when a patient suffers from more than one form of urinary incontinence, e.g. stress incontinence and urge incontinence.

The medical need is high for effective pharmacological treatments especially for mixed incontinence and stress urinary incontinence (SUI). This high medical need is a result of lack of efficacious pharmacological therapy coupled with high patient numbers. Recent estimates put the number of people suffering from SUI in the USA at 18 million, with women predominantly affected.

Stress incontinence may be confirmed by observing urine loss coincident with an increase in abdominal pressure, in the absence of a bladder contraction or an over distended bladder. The condition of stress incontinence may be classified as either urethral hypermobility or intrinsic sphincter deficiency. In urethral hypermobility, the bladder neck and urethra descend during cough or strain and the urethra opens with visible urinary leakage (leak point pressure between 60-120 cm H₂O). In intrinsic sphincter deficiency, the bladder neck opens during bladder filling without bladder contraction. Visible urinary leakage is seen with minimal or no stress. There is variable bladder neck and urethral descent, often none at all, and the leak point pressure is low (<60 cm H₂O). (J. G. Blaivas, 1985, Urol. Clin. N. Amer., 12:215-224; D. R. Staskin et al., 1985, Urol. Clin. N. Amer., 12:271-278).

Urge incontinence is defined as the involuntary loss of urine associated with an abrupt and strong desire to void. Although involuntary bladder contractions can be associated with neurologic disorders, they can also occur in individuals who appear to be neurologically normal (P. Abrams et al., 1987, Neurol. & Urodynam., 7:403-427).

Common neurologic disorders associated with urge incontinence are stroke, diabetes, and multiple sclerosis (E. J. McGuire et al, 1981, J. Urol., 126:205-209). Urge incontinence is caused by involuntary detrusor contractions that can also be due to bladder inflammation and impaired detrusor contractility where the bladder does not empty completely.

Overflow incontinence is characterized by the loss of urine associated with over distension of the bladder. Overflow incontinence may be due to impaired bladder contractility or to bladder outlet obstruction leading to over distension and overflow. The bladder may be under active secondarily to neurologic conditions such as diabetes or spinal cord injury, or following radical pelvic surgery.

Another common and serious cause of urinary incontinence (urge and overflow type) is impaired bladder contractility. This is an increasingly common condition in the geriatric population and in patients with neurological diseases, especially diabetes mellitus (N. M. Resnick et al., 1989, New Engl. J. Med., 320:1-7; M. B. Chancellor and J. G. Blaivas, 1996, Atlas of Urodynamics, Williams and Wilkins, Philadelphia, Pa.). With inadequate contractility, the bladder cannot empty its content of urine; this causes not only incontinence, but also urinary tract infection and renal insufficiency. Presently, clinicians are very limited in their ability to treat impaired detrusor contractility. There are no effective medications to improve detrusor contractility. Although urecholine can slightly increase intravesical pressure, it has not been shown in controlled studies to aid effective bladder emptying (A. Wein et al., 1980, J. Urol., 123:302). The most common treatment is to circumvent the problem with intermittent or indwelling catheterization.

There are a number of treatment modalities for stress urinary incontinence. The most commonly practiced current treatments for stress incontinence include the following: absorbent products; indwelling catheterization; pessary, i.e., vaginal ring placed to support the bladder neck; and medication (Agency for Health Care Policy and Research. Public Health Service: Urinary Incontinence Guideline Panel. Urinary Incontinence in Adults: Clinical Practice Guideline. AHCPR Pub. No. 92-0038. Rockville, Md. U.S. Department of Health and Human Services, March 1992; M. B. Chancellor, Evaluation and Outcome. In: The Health of Women With Physical Disabilities: Setting a Research Agenda for the 90's. Eds. Krotoski D. M., Nosek, M., Turk, M., Brooks Publishing Company, Baltimore, Md., Chapter 24, 309-332, 1996). Exercise is another treatment modality for stress urinary incontinence. For example, Kegel exercise is a common and popular method to treat stress incontinence. The exercise can help half of the people who can do it four times daily for 3-6 months. Although 50% of patients report some improvement with Kegel exercise, the cure rate for incontinence following Kegel exercise is only 5 percent. In addition, most patients stop the exercise and drop out from the protocol because of the very long time and daily discipline required.

Another treatment method for urinary incontinence is the urethral plug. This is a disposable cork-like plug for women with stress incontinence. Unfortunately, the plug is associated with over 20% urinary tract infection and, unfortunately, does not cure incontinence.

Biofeedback and functional electrical stimulation using a vaginal probe are also used to treat urge and stress urinary incontinence. However, these methods are time-consuming and expensive and the results are only moderately better than Kegel exercise. Surgeries, such as laparoscopic or open abdominal bladder neck suspensions; transvaginal approach abdominal bladder neck suspensions; artificial urinary sphincter (expensive complex surgical procedure with 40% reversion rate) are also used to treat stress urinary incontinence.

Other treatments include intra-urethral injection procedures with exogenous injectable materials such as silicone, carbon-coated particles, Teflon, collagen, and autologous fat. Each of these injectables has its disadvantages. U.S. Pat. Nos. 5,007,940; 5,158,573; and 5,116,387 to Berg report biocompatible compositions comprising discrete, polymeric and silicone rubber bodies injectable into urethral tissue for the purpose of treatment of urinary incontinence by tissue bulking. Further, U.S. Pat. No. 5,451,406 to Lawin reports biocompatible compositions comprising carbon-coated particulate substrates that may be injected into a tissue, such as the tissues of and that overlay the urethra and bladder neck, for the purpose of treatment of urinary incontinence by tissue bulking. One concern or adverse consequence associated with methodologies or therapies of tissue bulking relates to the migration of solid particles in the bulking agents from the original site of placement into repository sites in various body organs and the subsequent chronic inflammatory response of tissue to particles that are too small. These adverse effects are reported in urology literature, specifically in Malizia, A. A., et al., “Migration and Granulomatous Reaction After Periurethral Injection of Polytef (Teflon),” JAMA, 251:3277-3281 (1984) and in Claes, H., Stroobants, D. et al., “Pulmonary Migration Following Periurethral Polytetrafluoroethylene Injection For Urinary Incontinence,” J. Urol., 142:821-822 (1989). An important factor in assuring the absence of migration is the administration of properly sized particles. If particles are too small, they may be engulfed by the body's white cells (phagocytes) and carried to distant organs or may be carried away in the vascular system and travel until they reach a site of greater constriction. Target organs for particulate deposition include the lungs, liver, spleen, brain, kidney, and lymph nodes. The use of small diameter particulate spheres and elongate fibrils in an aqueous medium having biocompatible lubricant have been disclosed in Wallace et al., U.S. Pat. No. 4,803,075. While these materials showed positive, short-term augmentation results, this result was short lived as the material had a tendency to migrate and/or be absorbed by the host tissue.

Collagen injections generally employ bovine collagen, which absorbs in 4-6 months, resulting in the need for repeated injections. A further disadvantage of collagen is that about 5% of patients are allergic to bovine source collagen and develop antibodies.

Autologous fat grafting as an injectable bulking agent has a significant drawback in that most of the injected fat is resorbed. In addition, the extent and duration of the survival of an autologous fat graft remains controversial. An inflammatory reaction generally occurs at the site of implant. Complications from fat grafting include fat resorption, nodules and tissue asymmetry.

Recent approaches with muscle cell injection therapy using engineered muscle-derived cells might offer alternative therapy for the treatment of incontinence, particularly, stress urinary incontinence and for the enhancement of urinary continence. Preferably, the muscle-derived cell injection can be autologous, so that there will be minimal or no allergic reactions. Myoblasts, the precursors of muscle fibers, are mononucleated muscle cells, which differ in many ways from other types of cells. Myoblasts naturally fuse to form post-mitotic multinucleated myotubes and therefore can be used for long-term expression and delivery of bioactive proteins (T. A. Partridge and K. E. Davies, 1995, Brit. Med. Bulletin, 51:123-137; J. Dhawan et al., 1992, Science, 254: 1509-1512; A. D. Grinnell, 1994, In: Myology. Ed 2, Ed. Engel A G and Armstrong C F, McGraw-Hill, Inc, 303-304; S. Jiao and J. A. Wolff, 1992, Brain Research, 575:143-147; H. Vandenburgh, 1996, Human Gene Therapy, 7:2195-2200).

The use of myoblasts to treat muscle degeneration, to repair tissue damage or treat disease is disclosed in U.S. Pat. Nos. 5,130,141 and 5,538,722. Also, myoblast transplantation has been employed for the repair of myocardial dysfunction (S. W. Robinson et al., 1995, Cell Transplantation, 5:77-91; C. E. Murry et al., 1996, J. Clin. Invest., 98:2512-2523; S. Gojo et al., 1996, Cell Transplantation, 5:581-584; A. Zibaitis et al., 1994, Transplantation Proceedings, 26:3294). The use of myoblasts for treating urinary incontinence is disclosed in U.S. Pat. No. 6,866,842. as well as Transplantation. 2003 Oct. 15; 76(7):1053-60; J Urol. 2001 January; 165(1):271. and Yokoyama T. J., Urology, 165:271-276, 2001. Application WO2004055174, discloses culture medium composition, culture method, and myoblasts obtained, and their uses. Soft tissue and bone augmentation and bulking utilizing muscle-derived progenitor cells, compositions and treatments is disclosed in WO0178754. Myoblast therapy for mammalian diseases is disclosed in U.S. Pat. No. 9,909,451.

Although, the cell therapy offers advantages over other injectables, it has major disadvantages. One of the biggest limitations associated with the use of myoblasts for the treatment of stress urinary incontinence is that myoblasts require extensive in vitro cultivation for 3-4 weeks to achieve cell numbers required for injection making this therapy very expensive and unaffordable to many patients.

In view of the above-mentioned limitations and complications of treating urinary incontinence and bladder contractility, new and effective alternative modalities in this area are needed in the art.

### SUMMARY OF THE INVENTION

The invention is a composition for the treatment of incontinence comprising viable muscle tissue fragments and a carrier. The composition contains at least one viable muscle tissue fragment having at least one viable cell that can migrate from the tissue fragment and onto the transplantation site to form a new tissue. The viable muscle tissue fragments may be obtained from autologous, allogeneic, or xenogeneic tissue. The carrier includes, but is not limited to physiological buffer solution, injectable gel solution, saline and water. The compositions are useful in the treatment of incontinence by injecting the composition into the urogentital tissue, such as urethra, urethral sphincter, and bladder for urinary incontinences and colorectal tissue, such as colon, rectum and colorectal sphincter for fecal incontinence.

### DETAILED DESCRIPTION

The viable muscle tissue fragments may be obtained from autologous, allogeneic, or xenogeneic tissue. In one embodiment, the viable muscle tissue fragments are obtained from autologous tissue. The muscle tissue is obtained under aseptic conditions. The viable muscle tissue can be obtained using any of a variety of conventional techniques, including biopsy or other surgical tissue removal techniques. Once the viable muscle tissue has been obtained, the tissue can then be fragmented under sterile conditions. In addition, the tissue can be fragmented in any standard cell culture medium known to those having ordinary skill in the art, either in the presence or absence of serum. The viable muscle tissue fragment size can be in the range of about 0.1 to about 3 mm³, but preferably the viable muscle tissue fragments size are about 0.1 to about 1 mm³.

The composition of the present invention also includes a carrier. The carrier is biocompatible and has sufficient physical properties to provide for ease of injection. The carrier includes, but is not limited to physiological buffer solution, injectable gel solution, saline and water. Physiological buffer solution includes, but is not limited to buffered saline, phosphate buffer solution, Hank's balanced salts solution, Tris buffered saline, and Hepes buffered saline. In one embodiment, the physiological buffer is Hank's balanced salts solution. The injectable gel solution may be in a gel form prior to injection or may gel and stay in place upon administration.

The injectable gel solution is comprised of water, saline or physiological buffer solution and a gelling material. Gelling materials include, but are not limited to proteins such as, collagen, elastin, thrombin, fibronectin, gelatin, fibrin, tropoelastin, polypeptides, laminin, proteoglycans, fibrin glue, fibrin clot, platelet rich plasma (PRP) clot, platelet poor plasma (PPP) clot, self-assembling peptide hydrogels, and atelocollagen; polysaccharides such as, pectin, cellulose, oxidized cellulose, chitin, chitosan, agarose, hyaluronic acid; polynucleotides such as, ribonucleic acids, deoxyribonucleic acids, and others such as, alginate, cross-linked alginate, poly(N-isopropylacrylamide), poly(oxyalkylene), copolymers of poly(ethylene oxide)-poly(propylene oxide), poly(vinyl alcohol), polyacrylate, monostearoyl glycerol co-Succinate/polyethylene glycol (MGSA/PEG) copolymers and combinations thereof.

In one embodiment, the composition further comprises microparticles. Microparticles are also referred to as microbeads or microspheres by one of skill in the art. The microparticles provide both a temporary bulking effect and a substrate on which the viable muscle tissue fragments may adhere and grow. The microparticles must be large enough so as to discourage local and distant migration once injected, yet small enough so as to be administered by a hypodermic needle. Thus, microparticles have a substantially round shape with an average transverse cross-sectional dimension in the range of about 100 to about 1,000 microns, preferably in the range of about 200 to about 500 microns. The microparticles are preferably formed from a biocompatible polymer. The biocompatible polymers can be synthetic polymers, natural polymers or combinations thereof. As used herein the term “synthetic polymer” refers to polymers that are not found in nature, even if the polymers are made from naturally occurring biomaterials. The term “natural polymer” refers to polymers that are naturally occurring. The biocompatible polymers may also be biodegradable. Biodegradable polymers readily break down into small segments when exposed to moist body tissue. The segments then either are absorbed by the body, or passed by the body. More particularly, the biodegraded segments do not elicit permanent chronic foreign body reaction, because they are absorbed by the body or passed from the body, such that no permanent trace or residual of the segment is retained by the body.

In one embodiment, the microparticle is comprised of at least one synthetic polymer. Suitable biocompatible synthetic polymers include, but are not limited to polymers of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarate), polyurethane, poly(ester urethane), poly(ether urethane), and blends and copolymers thereof. Suitable synthetic polymers for use in the present invention can also include biosynthetic polymers based on sequences found in collagen, laminin, glycosaminoglycans, elastin, thrombin, fibronectin, starches, poly(amino acid), gelatin, alginate, pectin, fibrin, oxidized cellulose, chitin, chitosan, tropoelastin, hyaluronic acid, silk, ribonucleic acids, deoxyribonucleic acids, polypeptides, proteins, polysaccharides, polynucleotides and combinations thereof.

For the purpose of this invention aliphatic polyesters include, but are not limited to, homopolymers and copolymers of monomers including lactide (which includes lactic acid, D-, L- and meso lactide); glycolide (including glycolic acid); epsilon-caprolactone; p-dioxanone(1,4-dioxan-2-one); trimethylene carbonate(1,3-dioxan-2-one); alkyl derivatives of trimethylene carbonate; and blends thereof. Aliphatic polyesters used in the present invention can be homopolymers or copolymers (random, block, segmented, tapered blocks, graft, triblock, etc.) having a linear, branched or star structure. In embodiments where the scaffold includes at least one natural polymer, suitable examples of natural polymers include, but are not limited to, fibrin-based materials, collagen-based materials, hyaluronic acid-based materials, glycoprotein-based materials, cellulose-based materials, silks and combinations thereof.

One skilled in the art will appreciate that the selection of a suitable material for forming the biocompatible microparticles depends on several factors. These factors include in vivo mechanical performance; cell response to the material in terms of cell attachment, proliferation, migration and differentiation; and optionally, biodegradation kinetics. Other relevant factors include the chemical composition, spatial distribution of the constituents, the molecular weight of the polymer, and the degree of crystallinity.

In another embodiment, a biological effector may be incorporated within the composition of the invention. The biological effectors, promote the healing and/or regeneration of the affected tissue (e.g. growth factors and cytokines), prevent infection (e.g., antimicrobial agents and antibiotics), reduce inflammation (e.g., anti-inflammatory agents), prevent or minimize adhesion formation, such as oxidized regenerated cellulose (e.g., INTERCEED and Surgicel®, available from Ethicon, Inc.) and hyaluronic acid, and suppress the immune system (e.g., immunosuppressants).

Biological effectors include, but are not limited to heterologous or autologous growth factors, matrix proteins, peptides, antibodies, enzymes, glycoproteins, hormones, cytokines, glycosaminoglycans, nucleic acids, analgesics. It is understood that one or more biological effectors of the same or different functionality may be incorporated within the composition.

Heterologous or autologous growth factors are known to promote healing and/or regeneration of injured or damaged tissue. Exemplary growth factors include, but are not limited to, TGF-β, bone morphogenic protein, growth differentiation factor-5 (GDF-5), cartilage-derived morphogenic protein, fibroblast growth factor, platelet-derived growth factor, vascular endothelial cell-derived growth factor (VEGF), epidermal growth factor, insulin-like growth factor, hepatocyte growth factor, and fragments thereof. Suitable effectors likewise include the agonists and antagonists of the agents noted above.

Glycosaminoglycans are highly charged polysaccharides, which play a role in cellular adhesion. Exemplary glycosaminoglycans useful as biological effectors include, but are not limited to heparan sulfate, heparin, chondroitin sulfate, dermatan sulfate, keratin sulfate, hyaluronan (also known as hyaluronic acid), and combinations thereof.

The biological effector may also be an enzyme such as, matrix-digesting enzymes, which facilitate cell migration out of the extracellular matrix surrounding the cells. Suitable matrix-digesting enzymes include, but are not limited to collagenase, chondroitinase, trypsin, elastase, hyaluronidase, peptidase, thermolysin, matrix metalloprotease and protease.

One of ordinary skill in the art will appreciate that the appropriate biological effector(s) may be determined by a surgeon, based on principles of medical science and the applicable treatment objectives. The amount of the biological effector included with the composition will vary depending on a variety of factors, including the given application, such as promoting cell survival, proliferation, differentiation, or facilitating and/or expediting the healing of tissue. The biological effector can be incorporated within the composition of viable muscle tissue fragments and carrier before or after the composition is administered to the area of tissue injury.

The composition for treating incontinence as described herein may be prepared by first obtaining a muscle tissue sample from a donor (autologous, allogeneic, or xenogeneic) using appropriate harvesting tools. The muscle tissue sample is then finely minced and divided into small fragments either as the tissue is collected, or alternatively, the muscle tissue sample can be minced after it is harvested and collected outside the body. In embodiments where the tissue sample is minced after it is harvested, the tissue samples can be weighed and then washed three times in phosphate buffered saline. Approximately 100 to 500 mg of tissue can then be minced into small fragments in the presence of a small quantity, for example, about 1 ml, of a physiological buffering solution, such as, phosphate buffered saline, or a matrix digesting enzyme, such as 0.2% collagenase in Ham's F12 medium. The muscle tissue is minced into fragments of approximately 0.1 to 1 mm³ in size. Mincing the tissue can be accomplished by a variety of methods. In one embodiment, the mincing is accomplished with two sterile scalpels cutting in parallel and opposing directions, and in another embodiment, the tissue can be minced by a processing tool that automatically divides the tissue into particles of a desired size. In one embodiment, the minced tissue can be separated from the physiological fluid and concentrated using any of a variety of methods known to those having ordinary skill in the art, such as for example, sieving, sedimenting or centrifuging. In embodiments where the minced tissue is filtered and concentrated, the suspension of minced tissue preferably retains a small quantity of fluid in the suspension to prevent the tissue from drying out. The suspension of viable muscle tissue fragments is combined with a carrier, as described herein, and optionally with microparticles and delivered to the site of tissue repair via injection. In addition, a biological effector may be added to the composition with or without microparticles prior to administration to the site of tissue repair.

Compositions as described herein are useful in the treatment of soft tissue. Soft tissue refers generally to extraskeletal structures found throughout the body and includes but is not limited to, periodontal tissue, skin tissue, vascular tissue, muscle tissue, fascia tissue, ocular tissue, pericardial tissue, lung tissue, synovial tissue, nerve tissue, kidney tissue, esophageal tissue, urogenital tissue, intestinal tissue, colorectal tissue, liver tissue, pancreas tissue, spleen tissue, adipose tissue, and combinations thereof. Preferably, the compositions as described herein are useful in the treatment of urogenital tissue, such as urethra, urethral sphincter, and bladder, esophageal tissue, such as esophagus and esophageal sphincter, and colorectal tissue, such as colon, rectum and colorectal sphincter. The compositions can also be used for tissue bulking, tissue augmentation, cosmetic treatments, therapeutic treatments, and for tissue sealing.

A non-limiting example of the preparation of a composition for the treatment of incontinence is as follows. A patient is prepared for tissue repair surgery in a conventional manner using conventional surgical techniques. The muscle tissue sample used to form the composition is obtained from the patient using conventional tissue harvesting tools and techniques. The muscle tissue sample is finely minced and divided into viable muscle tissue fragments having a particle size in the range of about 0.1 to about 3 mm³. The tissue is minced using a conventional mincing technique such as cutting with two sterile scalpels in opposing parallel directions. Between about 100 to 500 mg of tissue is minced in the presence of about 1 ml of a physiological buffering solution, the amount of tissue required depends on the extent of the tissue injury at the site of repair. The viable muscle tissue fragments are filtered and/or concentrated to separate the viable muscle tissue fragments from the physiological buffering solution. The viable muscle tissue fragments are concentrated by centrifugation. The viable muscle tissue fragments are then combined with Hank's balanced salts solution carrier and optionally with microparticles and injected into the tissue repair site. A kit can be used to assist in the preparation of the compositions. The kit includes a harvesting tool, a sterile container that houses a reagent for sustaining tissue viability, a processing tool, a carrier, and a delivery device. The harvesting tool is used to obtain the viable muscle tissue from the subject. The tissue may be placed in the sterile container containing the reagent for sustaining tissue viability. Suitable reagents for sustaining the viability of the tissue sample include but are not limited to saline, phosphate buffering solution, Hank's balanced salts, standard cell culture medium, Dulbecco's modified Eagle's medium, ascorbic acid, HEPES, nonessential amino acid, L-proline, autologous serum, and combinations thereof. The processing tool is used to mince the tissue into viable muscle tissue fragments, or alternatively, the harvesting tool can be adapted to collect the tissue sample and to process the sample into finely divided tissue particles. The carrier may be physiological buffer solution, injectable gel solution, saline or water as described herein and may optionally include microparticles. The delivery device allows deposition of the composition of the viable tissue fragments in a carrier into diseased tissues, for example adjacent to or surrounding the sphincter regions of the urethra.

### EXAMPLE 1

The efficacy of a novel therapy based on the application of a composition of viable muscle tissue fragments for the restoration of leak point pressure (LPP) in a rat model of stress urinary incontinence (SUI) was examined. Viable muscle tissue fragments were generated from skeletal muscles of male rats. A total of 24 female Lewis rats were randomly assigned to 1 of 3 groups (8 animals per group), namely continent animals, incontinent animals injected with carrier, and incontinent animals injected with carrier+viable minced tissue fragments. SUI was created in the latter 2 groups by bilateral pudendal nerve transection (PNT). One week post-surgery, treatment was administered to each animal group by an intraurethral injection. After 5 weeks LPP was measured at least 4 times in each rat and the mean was determined.

### Animal Care

The animals used in this study were handled and maintained in accordance with all applicable sections of the Final Rules of the Animal Welfare Act regulations (9 CFR), the Public Health Service Policy on Humane Care and Use of Laboratory Animals, the Guide for the Care and Use of Laboratory Animals. The protocol and any amendments or procedures involving the care or use of animals in this study was reviewed and approved by the Testing Facility Institutional Animal Care and Use Committee prior to the initiation of such procedures.

Lewis rats were chosen due to their syngeneic phenotype. It allows evaluation of a composition for treatment of SUI derived from one rat and implanted into another without the use of immunosupression. The animals were individually housed in microisolators. Environmental controls were set to maintain temperatures of 18° C. to 26° C. (64° F. to 79° F.) with a relative humidity of 30% to 70%. A 12-hour light/12-hour dark cycle was maintained, except when interrupted to accommodate study procedures. Ten or greater air changes per hour with 100% fresh air (no air recirculation) was maintained in the animal rooms. Purina Certified Diet and filtered tap water was provided to the animals ad libitum.

### Materials and Methods

Animals. SUI was created by the previously established method of bilateral pudendal nerve transection (PNT). All procedures were performed under aseptic conditions. The rats were prepared for aseptic surgery and anesthesia was induced using isoflurane at 2.5%-4%. After induction, anesthesia was maintained with isoflurane delivered through a nose cone at 0.5-2.5%. For PNT surgery, the hair over the region spanning from the hips to the base of the tail, over the rump and down the back of the hind legs was shaved and the animal positioned in ventral recumbency. Via a dorsal longitudinal incision, the ischiorectal fossa was opened bilaterally. Using loop magnification the pudendal nerve was isolated and transected. The incision was closed using Nexaband® liquid topical tissue adhesive. The continent animal group had undergone the same surgical procedure with the exception of actually transecting the nerve.
Composition preparation and administration. Three male Lewis rats were euthanized with an overdose of intravenous pentobarbital sodium (100 mg/kg). Both of their quadricep tissue was removed. A piece of skeletal muscle was finely minced into fragments with a scalpel and then applied to a 300-micrometer cell strainer. Fragments were forced through the mesh with a 10 mL syringe plunger. The underside of the filter was scraped with a scalpel blade and the resulting viable muscle tissue fragments were weighed out. A total of 1 g of viable muscle tissue fragments was resuspended in 3 mL of Hank's balanced salt solution (HBSS) without Ca²⁺ and without Mg²⁺ (cat#:14175-095 Invitrogen, CA) into a uniform composition. The total tissue concentration was 0.3 g/mL. The viable minced muscle tissue suspended in HBSS was loaded into a 100 microliter Hamilton syringe and injected into the rat urethra with a hypodermic needle. Animals underwent treatment one-week post SUI injury creation. The female rats were anesthetized and then two injections (10 microliters each) per rat were performed at the 2-o'clock and 10-o'clock positions of the urethra. The carrier treated animals received injections of HBSS alone in the same manner.
Leak Point Pressure (LPP) Testing. At 5 weeks post-surgery, the rats were anesthetized and placed supine at the level of zero pressure and the bladder emptied manually. Subsequently the bladder was filled with saline solution at room temperature (5 ml per hour) through a suprapubic catheter. The suprapubic catheter was connected to a syringe pump and a pressure transducer. All bladder pressures were referenced to air pressure at bladder level. Pressure and force transducer signals were amplified and digitized for computer data collection using AD instruments, Power Lab computer software at 10 samples per second.

Peak bladder pressure was generated by slowly and manually increasing abdominal pressure until a leak occurred, at which point external abdominal pressure was rapidly released. LPP testing was performed a minimum of four times in each rat. The bladder was emptied using the Credé maneuver and refilled between LPP measurements. LPP values were acquired using an AD Instruments pressure transducer and analyzed using Power Lab Chart™ computer software. Individual outliers within LPP testing sessions for each animal were qualitatively identified as pressure artifacts and excluded from the study. Artifact pressure results were defined as pressure values (mmHg) that were considered artificially high or low compared to the other pressure results from the same LPP testing session. During LPP testing pressure artifacts can be generated in multiple ways including; inadvertently obstructing the catheter tip against either the mucosal wall of the bladder or urethra, the bladder not being completely evacuated of urine and/or saline, the animal being light on anesthetics during testing resulting in the animal contracting its bladder.

### Results and Discussion

The average LPP and standard deviation are reported below.

| Treatment | Number of | Average LPP | Standard |
|---|---|---|---|
| Group | animals | (mm Hg) | Deviation |
| Continent | 4 | 42.6 | 5.4 |
| animals | | | |
| Incontinent | 8 | 22.9 | 3.1 |
| animals injected | | | |
| with carrier | | | |
| Incontinent | 7 | 28.8 | 3.0 |
| animals injected | | | |
| with carrier + | | | |
| viable muscle | | | |
| tissue fragments | | | |

### CONCLUSIONS

The data indicates that a functional improvement was observed after four weeks in incontinent animals treated with viable muscle tissue fragments as compared to the incontinent animals injected with carrier alone. The improvement achieved was approximately 68% of continent animals, which indicates 42% improvement over incontinent animals injected with carrier alone. The data indicates that viable muscle tissue fragments produced a visible improvement over vehicle treatment and therefore can be a therapy for the treatment of stress urinary incontinence.

### EXAMPLE 2

The efficacy of a novel therapy based on the application of a composition of viable muscle tissue fragments for the restoration of leak point pressure (LPP) in 2 rat models of stress urinary incontinence (SUI) can be examined side by side. Viable muscle tissue fragment compositions can be prepared as described in Example 1. The 2 different rat models that can be compared are incontinent animals resulting from bilateral pudendal nerve trans section and from urethrolysis. Urethrolysis model will be created by a previously established method. Briefly, the animals will be anesthetized with an intraperitoneal injection of ketamine (60 mg/kg body wt) and xylazine (5 mg/kg body wt). They will be placed supine on a water-circulating heating pad. The abdomen will be prepped and draped in standard surgical fashion. A lower abdominal midline incision will be made, and the bladder and urethra will be identified. The proximal and distal urethra will be detached circumferentially by incising the endopelvic fascia and detaching the urethra from the anterior vaginal wall and pubic bone by sharp dissection. Care will be taken not to injure the ureters or compromise the inferior vesical vasculature. A cotton swab will be put into the vagina to aid with the dissection. The rectus fascia and skin will be closed with 4-0 polyglactin (Vicryl) and 4-0 Nylon sutures, respectively.

There will be 3 groups per injury model and rats can be randomly assigned to 1 of 3 groups namely continent animals, incontinent animals injected with carrier, and incontinent animals injected with carrier+viable muscle tissue fragments. One week post-surgery, treatment can be administered to each animal group by an intraurethral injection. After 5 weeks LPP can be measured 5 or 6 times in each rat and the mean can be determined.

### EXAMPLE 3

Description of various routes of administration of the composition into the urethra.

Periurethral route of minced tissue injection. Dispense the minced tissue composition containing microparticles into the special high-pressure syringe connected to a 17-gauge needle. Slowly insert the needle next to the urethral opening and into the submucosal tissues. After ascertaining the proper position of the needle, inject the suspension at 3 places around the urethra: the 2-, 6-, and 10-o'clock positions. As the injection progresses, the urethral lumen can be observed closing, and then the opening disappears. To assure success, visualize complete apposition (ie, kissing) of the urethral mucosa at the end of the procedure. One or 2 tubes may be injected to produce complete closure of the urethra.
Transurethral route. Using a special needle, inject minced tissue composition under direct vision underneath the urethral mucosa. Insert the cystoscope into the mid urethra. Under cystoscopic vision, carefully insert the tip of the needle underneath the urethral mucosa. Precisely deposit the minced tissue into the submucosal tissues until complete coaptation of the urethral mucosa is visualized.
Antegrade route. The antegrade route is reserved for males who are incontinent postprostatectomy. Create a suprapubic tract under adequate anesthesia. General anesthesia is preferred. Insert a flexible cystoscope into the bladder via the suprapubic tract. Identify the bladder neck. Under cystoscopic vision, carefully insert the tip of the needle underneath the bladder neck mucosa. Precisely deposit the minced tissue formulation into the submucosal tissues until complete coaptation of the bladder neck is noted.

### EXAMPLE 4

Rats are rendered incontinent by a validated model of urinary incontinence. Skeletal muscle biopsies can be harvested from skeletal muscles of rats (for example bicep, tricep or quadriceps) and finely minced into 0.1-0.4 mm³ fragments. The viable tissue fragments can be combined with a required volume, of carrier such as phosphate buffered saline (PBS) or HBSS or other carrier such as aqueous collagen solution, aqueous hyaluronic acid solution and microcarrier such as poly(glycolic acid) (PGA) or poly(lactic acid) (PLA). The process of mixing is followed by an immediate injection into the mid-urethra or the bladder neck of incontinent animals. At baseline and 3-4 weeks post-op, all of animals can undergo urodynamic testing. Urethral tissue can be harvested for organ bath isometric studies to test urethral function and for immunochemistry.

### EXAMPLE 5

The objective is to show that in pigs, autologous viable muscle tissue fragments from skeletal muscles (<1 mm in size) can be harvested, mixed with a carrier (PBS, HBSS, aqueous collagen solution, aqueous HA solution) and injected under sonographic control into the urethra. In addition, this procedure can be used to evaluate the composition as described herein as a therapeutic approach to treat urinary incontinence especially stress urinary incontinence. Skeletal muscle samples can be obtained through an open-incision biopsy. Approximately 100-500 mg of muscle tissue can be obtained from each pig. Samples are finely minced into <1 mm³ fragments. The viable muscle tissue fragments can be combined with a carrier and/or microparticles. With the help of transurethral ultrasound probe and injection system, samples can be injected into the rhabdosphincter and the urethral submucosa. Urethral pressure profiles can be measured before and after injection to determine the postoperative changes of urethral closure pressures. Histology can also performed on specimens obtained from pigs post-operatively.

### EXAMPLE 6

Purpose: The purpose of this experiment is to evaluate compositions of viable muscle tissue fragments for treatment of stress urinary incontinence. The viable muscle tissue fragments were characterized in terms of size, cell viability and ease of administration through various gauge needles.

### Method

A piece of rat skeletal muscle taken from a quadricep (approximately 1 g) is finely chopped with a scalpel and then applied to a 300 micrometer cell strainer. Viable muscle tissue fragments are forced through the strainer with a 10 mL syringe plunger. The fragments are washed with 30 mL of PBS and the suspension is pelleted by centrifugation at 1600 rpm for 5 minutes. Pellets is resuspended in 500 microliters of PBS and further characterized.

Average size distribution may range from 100-300 micrometers (approximately 0.1-1 mm³). Occasionally, long fragments (>1 cubic mm³) may be observed.

The ease of injection of the composition through various-gauge needles is also tested. Three gauge sizes are tried: 18, 21 and 25. The tissue fragment suspension will easily pass through all the needles even the 25-gauge size. Furthermore, no clumping/blockage will be observed. Composition samples will also be analyzed under microscope after every pass-through the needle and no disturbance/erosion of the mixture will be observed suggesting that the tissue fragments experienced unobstructed flow.

### EXAMPLE 7

Skeletal muscle or tissue biopsies from a relevant source can be harvested as detailed in previous examples. The biopsied tissue can be minced to a fine paste to form viable muscle tissue fragments. Fragments can be combined with a required volume of carrier and optionally microparticles as detailed in previous examples and can be injected into the internal or external anal sphincters using techniques known in the art for the treatment of fecal incontinence.

### EXAMPLE 8

Skeletal muscle or tissue biopsies from a relevant source can be harvested as detailed in previous examples. The biopsied tissue can be minced to a fine paste to form viable muscle tissue fragments. Fragments can be combined with a required volume of carrier and optionally microparticles as detailed in previous examples and using techniques known in the art can be injected into the lower esophageal sphincter and or the pyloric sphincter for the treatment of acid reflux and other digestive system related ailments.

### EXAMPLE 9

Fresh sample of porcine skeletal muscle was procured from Farm-to-Farm (Warren, N.J.). Samples were manually minced with a pair of scalpels. Minced skeletal muscle tissue was further fragmented by pushing through either a 300 (L3-50, ATM Products) or 425 (L3-40, ATM Products) micrometer steel mesh sieve. This process further minced the tissue to a more uniform size. Samples of each size were weighed out and set up at the following amounts: 10, 20, 30 and 40 micrograms. Minced tissue viability was determined by MTS assay (CellTiter 96® AQᵤₑₒᵤₛ One Solution Cell Proliferation Assay, Promega, Madison, Wis.) performed according to protocol provided by the manufacturer. Standard curve was also generated utilizing cells isolated from porcine skeletal muscle. Table 1 shows results of this assay.

TABLE 1
Results of MTS assay performed on various size and amounts of
minced porcine skeletal muscles.

| | Amount | |
|---|---|---|
| size | tested in μg | Cell count |
| 300 um | 10 | 24558 ± 3547 |
| | 20 | 62929 ± 2306 |
| | 30 | 79137 ± 6216 |
| | 40 | 105588 ± 2904 |
| 425 um | 10 | 21087 ± 923 |
| | 20 | 63646 ± 1364 |
| | 30 | 86824 ± 14785 |
| | 40 | 110533 ± 978 |
| MT | 10 | 22654 ± 948 |
| | 20 | 40591 ± 652 |
| | 30 | 58339 ± 468 |
| | 40 | 74637 ± 978 |

As can be seen, mincing process maintains skeletal muscle tissue viability. The viability is not altered by passing through a metal sieve to control minced fragments size.

Minced skeletal muscle tissue viability was further assessed over time in Hank's Balanced Salt Solution carrier (HBSS, Invitrogen, Carlsbad, Calif.) at 4° C. and at room temperature. Three quantities of tissue were investigated: 5 micrograms, 10 micrograms and 20 micrograms for up to 4 hours. In all cases samples were incubated either on ice (4° C.) or at room temperature (RT). Testing method employed was MTS assay (Promega). Table 2 shows results of this experiment.

TABLE 2
Results of MTS assay performed for the minced tissue viability study
for up to 4 hours.

| condition | T = 0 hours | T = 1 hours | T = 2 hours | T = 4 hours |
|---|---|---|---|---|
| 5 μg RT | 16132 ± 970 | 8330 ± 602 | 8356 ± 1476 | 2261 ± 331 |
| 10 μg RT | 33384 ± 1182 | 12088 ± 1297 | 19680 ± 7331 | 11462 ± 4296 |
| 20 μg RT | 49093 ± 742 | 28880 ± 1348 | 25422 ± 2544 | 11866 ± 6451 |
| 5 μg 4° C. | 16132 ± 970 | 16107 ± 1770 | 12992 ± 2939 | 5667 ± 1118 |
| 10 μg 4° C. | 33384 ± 1182 | 17504 ± 4999 | 18013 ± 786 | 16003 ± 1200 |
| 20 μg 4° C. | 49093 ± 742 | 22551 ± 12023 | 28799 ± 1892 | 29043 ± 3346 |

### Discussion

Tissue viability decreased with time. The best viability was recorded at time=0. However only minor changes in viability were recorded between 1 and 2 hrs. Slightly better viability was obtained at 4° C.

### CONCLUSION

This experiment emphasizes that tissue should be minced quickly taking less than 1 hour of total processing. Viability was also improved slightly with reduced temperature of 4° C.

### EXAMPLE 10

Characterization of cells grown out of the minced muscle tissue explants. Fresh sample of porcine skeletal muscle was procured from Farm-to-Farm (Warren, N.J.). Samples were manually minced with a pair of scalpels. Tissue fragments were cultured in either DMEM (Invitrogen, Carlsbad, Calif.), 10% FBS (Hyclone, Logan, Utah), penicillin/strepromycin (Invitrogen, Carlsbad, Calif.) or EGM-2 (Lonza, Walkerville, Md.) media. Cells that have grown out from porcine skeletal muscle explants in either DMEM (Invitrogen, Carlsbad, Calif.), 10% FBS (Hyclone, Logan, Utah), penicillin/strepromycin (Invitrogen, Carlsbad, Calif.) or EGM-2 (Lonza, Walkerville, Md.) media were phenotypically characterized by antibody staining and analyzed using a Guava instrument (Guava Technologies, Inc., Hayward, Calif.). Myoblasts were identified by CD56⁺ (N-cam, Abcam, Cambridge, Mass.) populations and endothelial cells were identified by a double positive CD34⁺/CD144⁺ (BD Pharmingen, San Jose, Calif., eBiosciences, San Diego, Calif. respectively) populations. As controls human derived skeletal muscle and endothelial cells were used. Table below summarizes the results of this experiment.

| | Cells grown in | Cells grown | Skeletal muscle | Endothelial cell |
|---|---|---|---|---|
| Marker | DMEM | in EGM-2 | cell control | control |
| CD34⁺ | <1% | <1% | <1% | 9% |
| CD56⁺ | 97% | 21% | 75% | <1% |
| CD144⁺ | <1% | <1% | <1% | 99% |

### Discussion

As can be observed from the table, the phenotype of cells migrating out from tissue fragments is dependent on the culture medium. While skeletal myoblasts constituted 97% of cell population grown out from explants in DMEM, 10% FBS, which is typically the growth medium designed for myoblasts. This percentage of myoblasts was decreased to 21% in EGM-2 medium. Since the remaining 79% of cells did not stain positive for endothelial markers we are postulating that the remaining cells are fibroblasts. Similar cell populations were obtained by Hannes Strasser et al. as well as other investigators who demonstrated that both myoblasts and fibroblasts are two major cell types within skeletal muscle tissue (Lancet 2007, 369:2179-86).

### CONCLUSION

In in vitro cell culture, we determined that at least some of the cells that grew out of the minced muscle tissue fragments were myoblasts, however these results are medium dependent. The presence of myoblasts in the minced tissue is advantageous for a regenerative therapy for treatment of SUI.

### EXAMPLE 11

### Porcine Urethral Cell Isolation

Porcine urethras were procured from Farm-to-Pharm (Warren, N.J.). Urethras were trimmed of fat and connective tissue and finely minced with a pair of scalpels. The weight of tissue was recorded (13.1 g) and tissue was placed in a 50 ml conical tube in a cocktail of digestion enzymes (see below) in DMEM (Invitrogen, Carlsbad, Calif.), 10% FBS (Hyclone, Logan, Utah), penicillin/streptomycin (Invitrogen, Carlsbad, Calif.).

The tube was wrapped with Parafilm M® to seal. The tube was transferred to 37° C. incubator shaking at 225 RPM for 2 hours. The completeness of digestion was checked every hour of incubation by removing the tube from the incubator and standing the tube upright for 1-2 minutes. When digestion was complete (no more than 2 hrs) the tube was stood upright for 1-2 minutes to allow large fragments to settle. The cell suspension (without the large fragments) was transferred to a new conical tube and diluted with fresh DMEM, 10% FBS, penicillin/streptomycin. Cell suspension was centrifuged at 150×g for 5 min and supernatant aspirated. Fresh medium was added (up to 50 ml in total volume) and resuspended. Cell suspension was centrifuged at 150×g for 5 min and supernatant removed. Fresh medium was added (up to 30 ml in total volume) and cells resuspended using a pipette by pipetting up and down. Resuspended cell pellet was filtered through a 100 μm filter. Cell suspension was centrifuged at 150×g for 5 min the supernatant aspirated and cell pellet resuspended in PBS. Cells were counted with the GUAVA® cell counter (Guava Technologies, Inc, Hayward, Calif.). Total of 6×10⁶ cells was obtained. Cells were plated in EGM-2 (Lonza, Walkersville, Md.) at 5,000 cells/cm² and placed in an incubator at 37° C.

### Digestion Enzymes

Collagenase 0.25 U/ml (Serva Electrophoresis, GmbH, Heidelberg, Germany), 2.5 U/ml dispase (Dispase 11165859, Ruche Diagnostics Corporation, Indianapolis, Ind.) and 1 U/ml hyaluronidase (Vitrase, ISTA Pharmaceuticals, Irvine, Calif.).

### Proliferation Assay

To assess the effect of minced porcine muscle tissue on the proliferation of cells isolated from porcine urethra. Urethra cells (isolated according to the method described above) were seeded onto 24-well dishes at a density of 10,000 cells/well. Experimental conditions were:
Low serum (20% of growth media)
Low serum (20% of growth media)+different amounts of minced tissue (500, 250, or 50 micrograms/well)
   Minced tissue was added to the inside of transwells (0.4 micron pore size). Two media types were tested—EGM-2 and DMEM/EGM-2 (50/50, vol/vol). At 2, 3 and 7 days, cells were harvested to obtain cell number and viability using the Guava instrument.

### Results:

| | | |
|---|---|---|
| day 2 | day 3 | day 7 |

EGM-2

| | | | |
|---|---|---|---|
| control | 7655 ± 370 | 5754 ± 1772 | 2167 ± 2254 |
| MT 500 | 9293 ± 107 | 8261 ± 1192 | 8119 ± 5741 |
| MT 250 | 11800 ± 854 | 10656 ± 1282 | 3672 ± 2393 |
| MT 50 | 10324 ± 2009 | 8569 ± 2088 | 3795 ± 4485 |

DMEM/EGM-2

| | | | |
|---|---|---|---|
| control | 17444 ± 1947 | 20786 ± 4198 | 123 ± 87 |
| MT 500 | 17972 ± 4265 | 26062 ± 1331 | 795 ± 355 |
| MT 250 | 19168 ± 4644 | 30875 ± 3289 | 568 ± 334 |
| MT 50 | 15166 ± 3688 | 25818 ± 4422 | 331 ± 43 |

### Discussion

Cells isolated from porcine urethra exhibited faster proliferation rates after two and three days of co-culture with minced muscle tissue than when incubated in the basal medium. The rate of proliferation was dependent on the basal medium, however there was a clear effect of the minced muscle tissue on further proliferation rate of cells isolated from porcine urethras. The effect was most pronounced at 3 days of culture after which time it tapered off presumably due to lack of fresh nutrients and presence of culture waste products. The greatest effect was noticed with 250 micrograms/well of minced muscle tissue, which produced a 77% and 93% increase in the proliferation rate of urethra-derived cells after 2 and 3 days respectively in EGM-2 and a 74% increase in the proliferation rate of urethra-derived cells after 3 days in DMEM/EGM-2 medium.

### CONCLUSION

The above-presented data clearly indicates that minced muscle tissue fragments have a positive in vitro effect on the proliferation rate of porcine urethra-derived cells. This suggests that at least partially, the mechanism of action of these cells responsible for restoration of leak point pressure (LPP) in incontinent rats (presented in Example 1), is increase in healthy cells and therefore regeneration of urethral tissue. This also suggests that their therapeutic effect is not just a bulking action but rather a trophic effect, which promotes bona fide long-term regenerative response.

### EXAMPLE 12

### Introduction

The objective of the study was to determine the safety of the test article and also to record the functional changes in urodynamics and histological changes in the female porcine urethra induced by the injection of autologous tissue-derived products into the muscular wall surrounding the urethral lumen up to a period of three months after injection in a healthy animal.

This study was performed in compliance with the Food and Drug Administration Good Laboratory Practice Regulations, Title 21 of the U.S. Code of Federal Regulations, Part 58, issued Dec. 22, 1978 (with all applicable revisions). All changes or revisions to the approved protocol are maintained with the original protocol in the study file.

### Experimental Design

Seven (plus 1 spare) animals were studied over a maximum of 3 months +/−5 days post treatment. Pre-Treatment procedure were performed a minimum of 7 days prior to treatment. Animals in both groups were implanted with indwelling bladder catheters (Day ≧7). Exception was the spare animal. Day 0 dosing injections: the Test animals received the autologous tissue derived products generated from the muscle donation. The Control animals received injections of the Vehicle (Hanks Balanced Salt Solution—HBSS—Invitrogen) article. Animals in test group underwent a muscle biopsy from each hind limb. Explanted tissue was processed on site to generate the Test Article used for treatment injection. Animals were recovered and survived for a period of approximately 3 months. Urodynamic assessment of the bladder was performed at designated time intervals at pre-treatment, day 21, 29, 57 and 94 post-treatment. The urodynamic testing included Leak Point Pressure (LPP) and Urethral Pressure Profile (UPP) measurements. All animals were euthanized ˜3 months post treatment and the urinary tract underwent microscopic evaluation.

### Quarantine

All animals received within the facility received a physical exam prior to release from quarantine on Day 6. Observed morphology and behavior were deemed within the norm, and animals were unconditionally released by the facility veterinarian.

### Treatment

Muscle Biopsy: Muscle biopsy to prepare test article was performed utilizing a 8 mm punch biopsy needle.
Test Article and Vehicle Preparation: Vehicle used for the study was Hanks Balanced Salt Solution (HBSS, Invitrogen, Carlsbad, Calif.). Test Article was prepared in the following way. Muscle biopsy was performed and between 500-700 mg of tissue was obtained. The tissue was trimmed of fat and finely minced with a pair of scalpels. Tissue was kept moist during the process by a small quantity of HBSS. Following mincing, tissue was applied to a 425 micrometer metal mesh strainer (L3-40, ATM Products) and further fragmented by passing through using a syringe plunger (5 cc). Sample was collected and resuspended in HBSS (1.5 ml total volume).
Treatment Procedure: Test and control article delivery was carried out under anesthesia at the urethral opening. Treatment procedure in all animals was altered to accommodate injection volumes to the available treatment area. The injections were performed circumferentially (6-8 injections per site) at 4 distinct places along the urethra between the caudal and middle third-away from the bladder neck with cystoscopic guidance.

### Leak Point Pressure Testing

LPP values were acquired using an AD Instruments pressure transducer and analyzed using Power Lab Chart™ computer software. Results were transcribed and tabulated (Table 3). LPP on Day 0 for all ported animals was performed using the indwelling urinary bladder catheter. Given that the UPP measurements were also to be done at the same time the port was ultimately not used in future LPP measurements.

### Maximum Urethral Pressure Testing

UPP values were acquired using an AD Instruments pressure transducer and analyzed using Power Lab Chart™ computer software. MUCP was then calculated according to standard methods. Results were transcribed and tabulated (Table 3).

### Necropsy/Tissue Collection/Histopathology:

After three months, the animals were euthanized and subjected to a limited necropsy and limited tissue collection consisting of the entire urinary tract. The urethra, urinary bladder, ureters and kidneys were collected at necropsy. The urethras were fixed with 10% neutral buffered formalin under pressure for a period of about 24 hours. After fixation, tissues were submitted to Vet Path Services, Inc. (VPS) for histological processing and histopathological examination. The urethra was trimmed, embedded in paraffin and sectioned. Microtome sections were taken at 2.5 mm intervals along the entire urethra starting at the bladder neck and stained with hematoxylin and eosin and Masson's Trichrome stains. Urethral measurements were performed on the Masson's Trichrome stained slides. Measurements were obtained with image analysis histomorphometry. The total thickness of the urethra, the thickness of the smooth muscle and skeletal muscle layers were obtained. The thickness of the connective tissue was obtained by subtracting the combined thickness of the smooth and skeletal muscle layers from the total thickness of the urethra.

### Results

### Urodynamics Testing

Results of LPP and mUCP testing are contained in Table 3.

TABLE 3
Results of LPP and mUCP testing for each animal in the study.

| Animal | | | | MUCP |
|---|---|---|---|---|
| Number | Group | Day | LPP mmHg | mmHg |
| 1 | Vehicle | 0 | 14.5 | 39.3 |
| | | 21 | 32.2 | 56.9 |
| | | 29 | 12.8 | 52.8 |
| | | 57 | 31.3 | 126.0 |
| | | 94 | 32.6 | 79.8 |
| 2 | Vehicle | 0 | 27.6 | 29.0 |
| | | 21 | 21.5 | 44.9 |
| | | 29 | 20.7 | 62.5 |
| | | 57 | 38.1 | 68.0 |
| | | 94 | 31.0 | 101.2 |
| 3 | Test article | 0 | 27.9 | 40.9 |
| | | 21 | 21.6 | 145.5 |
| | | 29 | 38.4 | 59.3 |
| | | 57 | 51.9 | 71.7 |
| | | 94 | 41.8 | 127.9 |
| 4 | Test article | 0 | 31.5 | 61.4 |
| | | 21 | 29.4 | 71.6 |
| | | 30 | 29.2 | 47.2 |
| | | 58 | 23.4 | 47.9 |
| | | 96 | 27.1 | 58.9 |
| 5 | Test article | 0 | N/A | 49.2 |
| | | 21 | 61.1 | 144.3 |
| | | 29 | N/A | 67.0 |
| | | 57 | 16.1 | 90.4 |
| | | 94 | 25.5 | 101.7 |
| 6 | Test article | 0 | 41.8 | 95.0 |
| | | 20 | 37.4 | 60.9 |
| | | 28 | 25.5 | 66.5 |
| | | 54 | 54.2 | 33.8 |
| | | na | na | na |
| 7 | Test article | 0 | 35.9 | 39.9 |
| | | 20 | 92.5 | 107.2 |
| | | 28 | 26.7 | 75.6 |
| | | 56 | 23.7 | 51.8 |
| | | 93 | 16.4 | 74.6 |

No differences were observed in LPP between control and test article animals. However, data suggest that a significant increase (>250%) in maximal urethral closure pressure (mUCP) was observed on day 21 in 3/5 test article animals. As time progressed, the mUCP values equilibrated with those of the control animals (see table 3). Note that test animal 6 had to be euthanized prior to day 93 due to an unrelated injury.

### Histological Observations

Vehicle control animals: Minimal to mild epithelial hyperplasia (2/2) and none to minimal chronic active and erosive inflammation (1/2) were seen in the urethral urothelium of control animals. Minimal or mild subacute inflammation was seen in the epithelium and lamina propria of both control animals. None to mild cysts (1/2) and edema (2/2) and none to minimal hemorrhage (2/2) and lymphoid nodule-like aggregates (2/2) were observed in the lamina propria of control animals. None to minimal subacute inflammation was seen in the tunica muscularis of (1/2) control animals. The mean epithelial hyperplasia score was 1.3, the chronic active and erosive inflammation score was 0.1, the subacute inflammation score in the epithelium and lamina propria was 1.7, the cyst score was 0.2, the edema score was 0.8, the hemorrhage score was 0.5 and the lymphoid aggregates score was 0.6. The mean subacute inflammation score in the muscularis was 0.1.
Test animals: None to mild epithelial hyperplasia was seen in the urethral urothelium of 5/6 test animals. Minimal or mild subacute inflammation was seen in the epithelium and lamina propria of 6/6 test animals. None to mild cysts (2/6) and edema (6/6) and none to minimal hemorrhage (5/6) and lymphoid nodule-like aggregates (6/6) were observed in the lamina propria of test animals. None to minimal subacute inflammation was seen in the tunica muscularis of 4/6 test animals. The mean epithelial hyperplasia score was 0.6, the subacute inflammation score in the epithelium and lamina propria was 1.3, the cyst score was 0.1, the edema score was 0.9, the hemorrhage score was 0.2 and the lymphoid aggregates score was 0.4. The mean subacute inflammation score in the muscularis was 0.1.

### Image Analysis Histomorphometry

Vehicle control animals: In the vehicle control animals, the average total thickness of the urethra was 1.4, the average thickness of the smooth muscle was 0.7, the average thickness of the skeletal muscle was 0.0 and the average thickness of the connective tissue was 0.8. The smooth muscle represented 47% of the thickness of the urethra and the striated muscle represented 0% of the thickness of the urethra.
Test animals: In the test animals, the average total thickness of the urethra was 1.7, the average thickness of the smooth muscle was 0.8, the average thickness of the skeletal muscle was 0.1 and the average thickness of the connective tissue was 0.9. The smooth muscle represented 45.0% of the thickness of the urethra and the striated muscle represented 3.2% of the thickness of the urethra.

### Discussion

At the time point of 21 days, a clear increase in maximal urethral closure pressures (mUCPs) could be observed in 3 out of the 5 test article animals. The two remaining animals did not respond to the injections in a similar way due to unknown reasons. A subsequent decrease in mUCPs was observed on days 28, 57 and 94. The exact mechanism leading to reduction of UPP is not clear. In fact, we did not see fibrosis or inflammation. Perhaps the fact that there was no injury created in the animals affected the results. Integration of the injected tissue into the tissue of the urethra in the test article group and the formation of new muscle fibers were seen in standard histological examination. Another important point of the histological evaluation is that no signs of infection, inflammation, or fibrosis could be detected in the specimens. Furthermore, there was no evidence for the formation of “bulks” of new tissue or tissue depots leading to compression or obstruction of the urethral lumen. Therefore the postoperative effect was not caused by simple obstruction or compression of the urethra.

### CONCLUSIONS

There was a significant (>250%) increase in mUCP in 3/5 test article animals at day 21 post-treatment. There was no evidence of any treatment-induced local irritation when examining the urethras. Urethras changes were relatively similar among test and vehicle control animals. The severity of epithelial hyperplasia and subacute inflammation in the epithelium and lamina propria were slightly lower in the test urethras compared to the control urethras. Chronic active and erosive inflammation was only seen in the vehicle control urethras. The average total thickness of the urethra was slightly higher in the test urethras compared to the control urethras. In the test urethras, the striated muscle represented 3.2% of the thickness of the urethra, but no striated muscle was observed in the vehicle control urethras. The safety study of the minced muscle fragments indicated that there was no significant adverse affects. The significant (>250%) increase in mUCP at day 21 as well as the evidence of striated muscle indicates that the minced muscle tissue is useful in treating SUI.

We claim:

1. A composition for the treatment of incontinence comprising viable muscle tissue fragments and a carrier.

2. The composition of claim 1 wherein the viable muscle tissue is selected from the group consisting of autologous tissue, allogeneic tissue, xenogeneic tissue, and a mixture thereof.

3. The composition of claim 1 wherein the carrier is selected from the group consisting of physiological buffer solution, injectable gel solution, saline and water.

4. The composition of claim 3 wherein the carrier is physiological buffer solution.

5. The composition of claim 4 wherein the physiological buffer solution is buffered saline, phosphate buffer solution, Hank's balanced salts solution, Tris buffered saline and Hepes buffered saline.

6. The composition of claim 3 wherein the carrier is an injectable gel solution comprising a physiological buffer and a gelling material.

7. The composition of claim 6 wherein the gelling material is selected from the group consisting of proteins, polysaccharides, polynucleotides, alginate, cross-linked alginate, poly(N-isopropylacrylamide), poly(oxyalkylene), copolymers of poly(ethylene oxide)-poly(propylene oxide), poly(vinyl alcohol), polyacrylate, monostearoyl glycerol co-Succinate/polyethylene glycol (MGSA/PEG) copolymers and combinations thereof.

8. The composition of claim 1 further comprising at least one microparticle.

9. The composition of claim 8 wherein the microparticle is comprised of a biocompatible polymer selected from the group consisting of synthetic polymers, natural polymers and combinations thereof.

10. A method of treating incontinence comprising injecting into a urogenital tissue the composition of claim 1.

11. A method of treating incontinence comprising injecting into a colorectal tissue the composition of claim 1.

12. A method of making a composition for the treatment of incontinence comprising the steps of:
a. providing at least one viable minced muscle tissue fragment; and
b. combining said fragment with a carrier suitable for injection into a urogenital tissue.
